(19) United States
(12) Patent Application Publication
(10) Pub. No.: US 2018/0228486 A1
(43) Pub. Date: Aug. 16, 2018

(54) SUTURING DEVICE AND METHOD OF USE

(71) Applicant: Beacon Surgical Instruments, LLC, Dobbs Ferry, NY (US)

(72) Inventors: Sundaram Ravikumar, Briarcliff Manor, NY (US); Guy Osborne, Dobbs Ferry, NY (US); Yaniv Ben-Asher, Dobbs Ferry, NY (US)

(73) Assignee: Beacon Surgical Instruments, LLC, Dobbs Ferry, NY (US)

(21) Appl. No.: 15/751,863

(22) PCT Filed: Aug. 11, 2016
(86) PCT No.: PCT/US16/46596

§ 371 Date: Feb. 11, 2018

Related U.S. Application Data
(60) Provisional application No. 62/203,867, filed on Aug. 11, 2015.

(51) Int. Cl. A61B 17/04

(52) U.S. Cl. CPC A61B 17/0469; A61B 17/0482; A61B 17/0467; A61B 17/12013; A61B 2017/0414; A61B 2017/0453; A61B 2017/0496; A61B 17/0401

(57) ABSTRACT

A suturing device for use with a tubular vessel, tissue or organ including a hollow housing, an activation mechanism and a flexible suture guide. The suturing device includes a suture which may be pre-knotted or include a crimp, anchor or other retaining means. The flexible suture guide of the suturing device is placed around the outer diameter of the tubular vessel, tissue or organ and the activation mechanism causes the suture to be placed around the outer diameter of the tubular vessel, tissue or organ and knotted. The suturing device may include a severing means which severs the suture at the knot.

### RELATED APPLICATIONS

This application claims priority from PCT Patent Application No. PCT/US2016/046,596 filed on Aug. 11, 2016 and provisional application Ser. No. 62/203,867 filed on Aug. 11, 2015.

### FIELD OF THE INVENTION

The present invention relates to a suturing device and method of use of the device.

### BACKGROUND OF THE INVENTION

The present invention relates to a suturing device. Suturing devices which may be used during laparoscopic surgery are known but have many disadvantages and problems. Certain devices include suture guns, staplers, clip appliers, sutures with fasteners, sutures with anchors and other known combinations. Some devices have simple handles while others have push rod mechanisms, thumb grip mechanisms and gun or pistol handle mechanisms. Certain mechanical suturing devices for suturing the skin are already known, and may be produced in the shape of a gun.

In use, a suturing gun device of the conventional kind is grasped by the surgeon with one hand, while he operates a forceps device with the other hand in order to clamp the margins of the wounded skin together and maintain them in this clamped position so that the stitches delivered by the suturing gun can grip both the wound margins, such that the wound is properly closed. This procedure is very toilsome for the surgeon and often, to facilitate this operation, the mechanical suturing device is grasped by the right hand of the surgeon and the clamping of the skin margins is carried out by an assistant who operates a pair of toothed forceps to hold the wound margins clamped until the stitches have been applied. The latter solution facilitates the surgeon's task but requires the presence of two operators and therefore increases overall cost of the operation as well as potential for increased tear of the port or incision point on the patient's fascia and compounds the risk for human error, mechanical error with multiple surgical instruments being used simultaneously or even subsequently, and other complications.

Further, in certain locations within the body, there is limited space in which to suture and operate more than one device. In such circumstance where passing a suture is difficult due to the limited space available to work in, devices and techniques have been developed to assist a person, such as a surgeon, in manipulating sutures. Some sutures are manipulated by plunger-type suture passing devices, whereby a surgeon squeezes a handle to move a plunger which projects a loop through a guide tip. The handle of such plunger-type devices must be squeezed to slide the plunger so that the loop is extended, thus requiring at least two fingers to operate the devices. In some cases, the plunger-type devices also have obtrusive handles, which can impair maneuverability of the device when working in confined spaces. For example, during surgery, a surgeon may utilize multiple devices all directed toward use in a small portion of the patient's body. The confined surgical work space can become cluttered with devices and thus impair maneuverability of individual devices. Therefore, a compact device would grant the surgeon greater ease of maneuverability in the confined surgical work space.

Further, in the known art, multiple devices must be employed when the target location for the suture is a tubular organ, tissue or vessel. Therefore a second device, third device or a greater number of devices must be employed to manipulate the suture once deployed around the target tubular organ, tissue or vessel. Again, at least two devices if not more must be employed at the same time or subsequent times during the surgical procedure to accomplish a suture.

In certain surgical procedures a tubular structure must be sutured which is difficult given the location of the suture and the shape of the tissue, organ or blood vessel being sutured. For instance, in suturing an appendix, cystic duct or cystic artery it is difficult to manipulate and maneuver a suture around the target location and tie, tightened and knot the suture. In such instances more than one suture device must be used if a stapler or clip applier is not employed. To suture a tubular target vessel, tissue or organ a surgeon must conventionally employ at least two surgical instruments or devices with one device containing or holding the suture and then typically a second surgical instrument or device, such as a grasper, is used to move the suture around the outer diameter of the tubular target vessel, tissue or organ and then the grasper is moved to a different location within the body cavity to pull the suture on the other side of the tubular target vessel, tissue or organ and then the suture is knotted or tied, again typically by manipulation of the grasper or other surgical instrument or device. This process and method is cumbersome in a small body cavity location and can lead to complications and errors such as without limitation nicking non-target vessel, tissue or organ by the grasper due to the limited space within the body cavity location.

Surgical staplers and clip appliers have many problems in general and when used to staple or clip a tubular target vessel, tissue or organ. For instance, surgical staplers must be easily dismantable for sterilization purposes while at the same time meet high standards of reliability, effectiveness, and precision in operation. All of these requirements increase the cost of surgical staplers which may have relatively complex parts and mechanisms. Still another requirement is that the surgical stapler function easily and without danger of failure as it is deploying surgical staples into a body cavity. A further requirement for conventional surgical staplers is that each stapler contains staples corresponding with the thickness of the tissue or target organ to be stapled. Thus, the surgical stapler is limited in use in that the staple size determines the target vessel, tissue or organ in which each surgical stapler may be employed to deploy the specific staple size. Without being bound by theory, it is proposed that the staple size is limited based on the squeezing of interfacing tissue or organ layers together within a limited range of pressing forces without causing injury to the actual target tissue or organ. Conversely, the final or bent-up shape of the surgical staple must be very accurately predetermined so as to provide positive and firm holding together of the interfacing target tissue or organ layers without, however, the staple itself unduly tearing or otherwise injuring the target tissue or organ layers. Thus, the surgical staplers known in the art have significant limitations in use, especially in regard to a tubular target vessel, tissue or organ. For tubular target vessel, tissue or organ stapling a surgical stapler has to be held with a certain force against the tubular target vessel, tissue or organ such that the target location is stapled without staling any other vessel, tissue or organ in the area (the non-targets). In some surgical methods this requires a second or third surgical instrument which holds the non-target vessel, tissue or organ away from the stapling area, again adding more devices within the small confines of a body cavity and possibility increasing the errors and complications during the surgery.

Although many of the above-discussed types of suturing devices and techniques are satisfactory in various applications using staples, dips or fasteners, there is a need to provide improved suturing devices which are relatively easier to use. In particular, there is a need for surgical suturing devices which do not require a second separate suture guide, graspers or forceps to manipulate the suture and which overcome the disadvantages associated with conventional surgical staples, clips and fasteners. There is a further need for one device which may be used on various target vessels, tissue or organs despite the varying thickness of the target suture location.

There is, therefore, a need for a new suture device in which a surgeon can suture completely around a tubular structure such as a target organ, tissue or vessel, with just one device. This allows the surgeon to more easily and steadily control the operation of the suture device, and thus the manipulation of the suture. There is also a need for a new suture device that has a compact handle portion thus allowing for greater maneuverability of a device within a confined surgical work space.

The present invention aims at obviating all the above mentioned disadvantages by providing a new suturing device which permits the suturing operation of a tubular object to be carried out by the surgeon with only one device (which is not a stapler, clip applier or fastener). The result obtains the advantages of better suturing around the tubular structure, facilitating the suturing operation and requiring less time for carrying out steps which can be performed simultaneously.

It is another object of the present invention to provide a new suturing device which permits the suturing operation to be carried out by the surgeon without the necessity of resorting to a second device or even an assistant.

It is another object of the present invention to provide a new suturing device with which is not dependent on the thickness of the area to be sutured or the diameter of a target object to be sutured, whether it is tubular or otherwise.

It is also an object of the invention to include a suturing device which sutures and cuts the suture in the same device.

These and other advantages and objects of the present invention will be discussed below.

### SUMMARY OF THE INVENTION

The object of the present invention is therefore to provide a suturing device for use in suturing around tubular structures.

Another object of the present invention is to provide a new suturing device which permits the suturing operation to be carried out by the surgeon without the necessity of resorting to a second device or even an assistant.

Another object of the present invention is to provide a new suturing device which is not dependent on the thickness of the area to be sutured or the diameter of a target object to be sutured, whether it is tubular or otherwise.

Another object of the present invention is to provide a suturing device which sutures and cuts the suture in the same device.

These objects are achieved by the present invention according to the preambles of the independent claims and provided with the features according to the characterizing portions of the independent claims.

Preferred embodiments of the present invention are set forth in the dependent claims.

### BRIEF DESCRIPTION OF THE DRAWINGS

FIG. 1 shows an embodiment of the present invention of the suturing device.

FIG. 2 is the distal end of one embodiment of the present invention of the suturing device.

FIG. 3 is a cross section the distal end of one embodiment of the present invention of the suturing device showing the outer coil tube and the inner coil tube with the flexible suture guide extending out of the hollow housing.

FIG. 4 is a cross section the distal end of one embodiment of the present invention of the suturing device with a suture holder mechanism.

FIG. 5 is a cross section the distal end of one embodiment of the present invention of the suturing device showing the outer coil tube, the inner coil tube and the flexible suture guide extending out of the hollow housing with the distal end of the flexible suture guide held within the aperture on the distal end cap.

FIG. 6 is the distal end of one embodiment of the present invention of the suturing device showing the flexible suture guide extending out of the hollow housing in a circular form with the distal end of the flexible suture guide held within the aperture on the distal end cap.

FIG. 7 is the proximal end of one embodiment of the present invention of the suturing device showing the hollow housing, pistol grip mechanism, the handle and the plunger, with the handle and plunger in an activated state.

FIG. 8 is the proximal end of one embodiment of the present invention of the suturing device showing the hollow housing, pistol grip mechanism, the handle and the plunger, with the handle and plunger and trigger means in an activated state.

FIG. 9 is a distal end view of another embodiment of the present invention of the suturing device with a tubular flexible suture guide.

FIG. 10 is a distal end view of another embodiment of the present invention of the suturing device with a flexible suture guide and further including a cutting means which is in an unactivated state within the hollow housing.

FIG. 11 is a close up view of the distal end of the suturing device as shown in FIG. 10 with the cutting means in an activated state extending out of the hollow housing.

FIGS. 12A through 12E are one embodiment of the method of the present invention using the inventive suturing device with a tubular vessel.

### DETAILED DESCRIPTION OF THE PREFERRED EMBODIMENTS

Reference will now be made in detail to the present invention, examples of which are illustrated in the accompanying drawings.

In accordance with one aspect of the invention, as indicated above, the inventive surgical instrument device is provided which has utility with various different body organs or tissue which may need to be sutured during open or laparoscopic surgery. The inventive surgical device may also be employed to suture tubular organs such as an appendix, cystic duct or cystic artery that is difficult to manipulate and maneuver a suture around the target location and tie, tightened and knot the suture within the one inventive device 100.

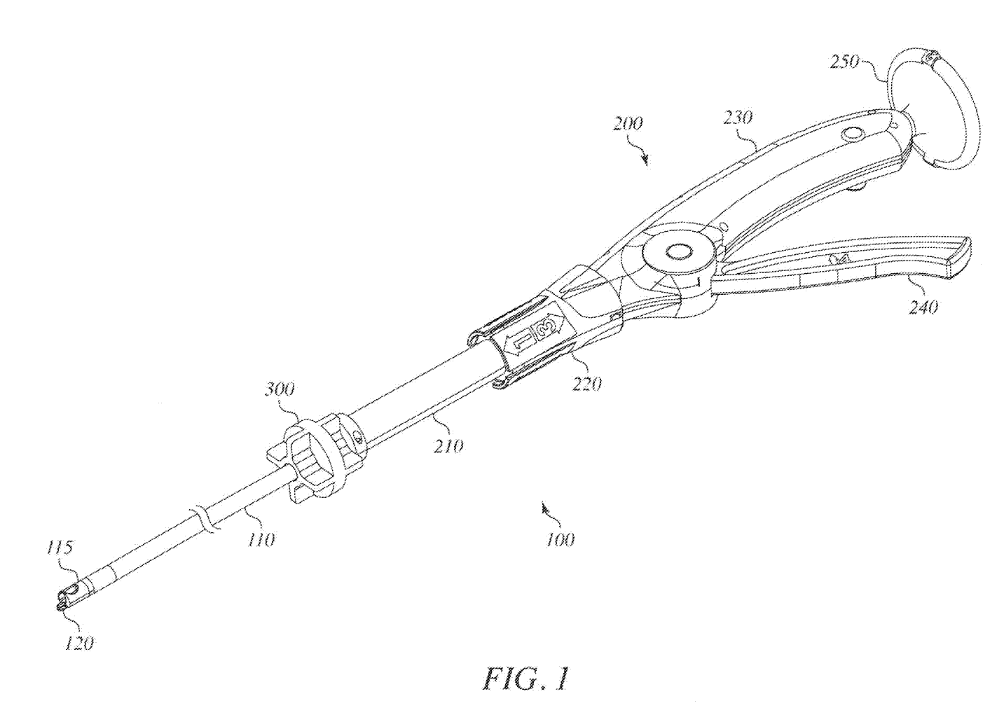

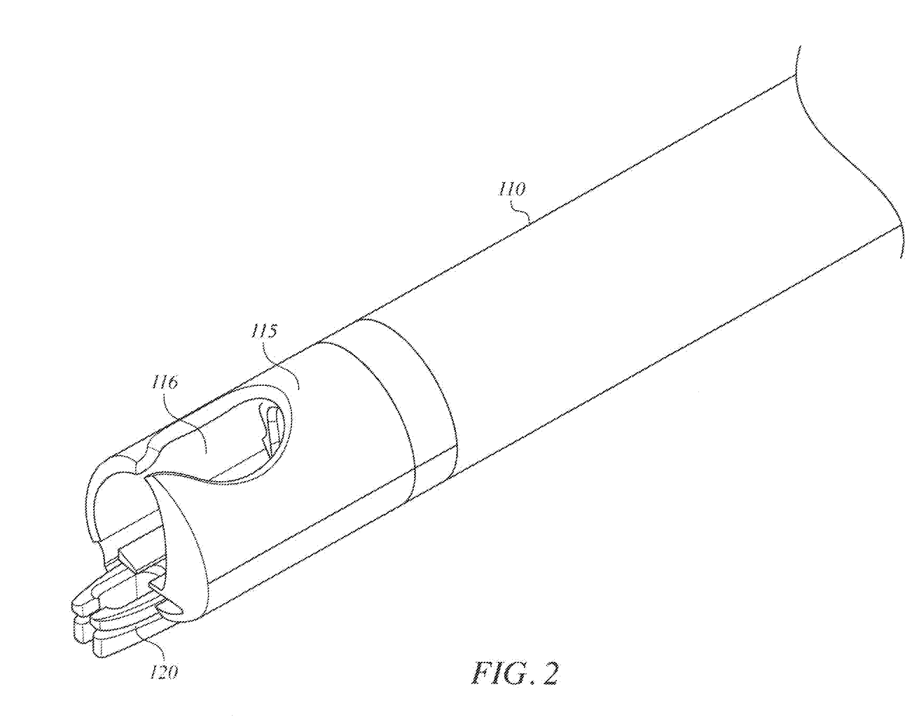

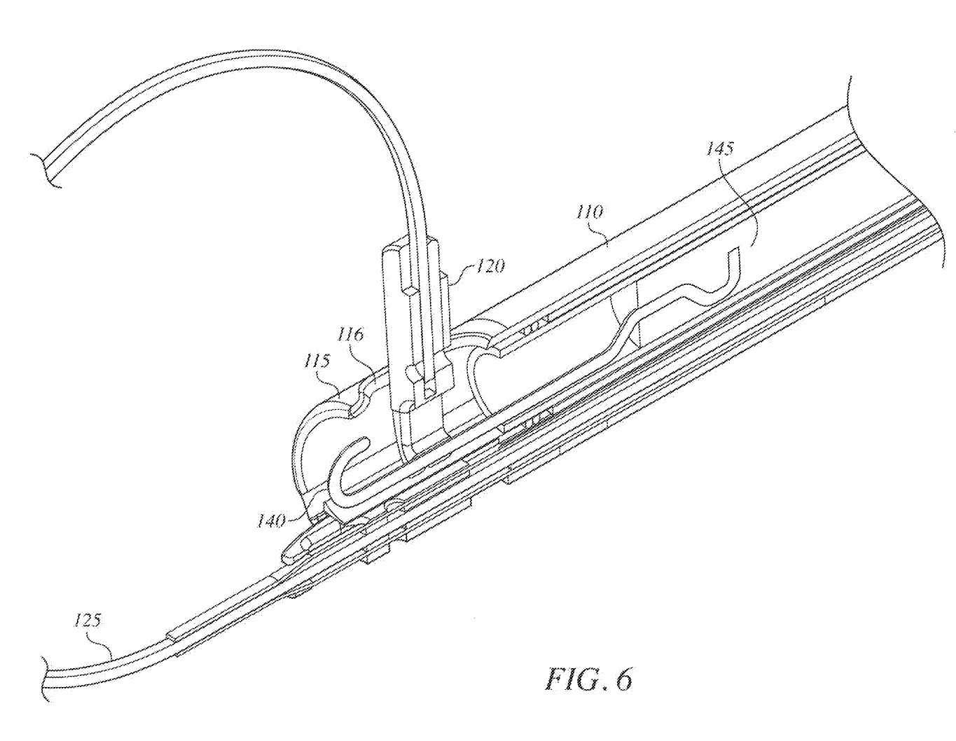

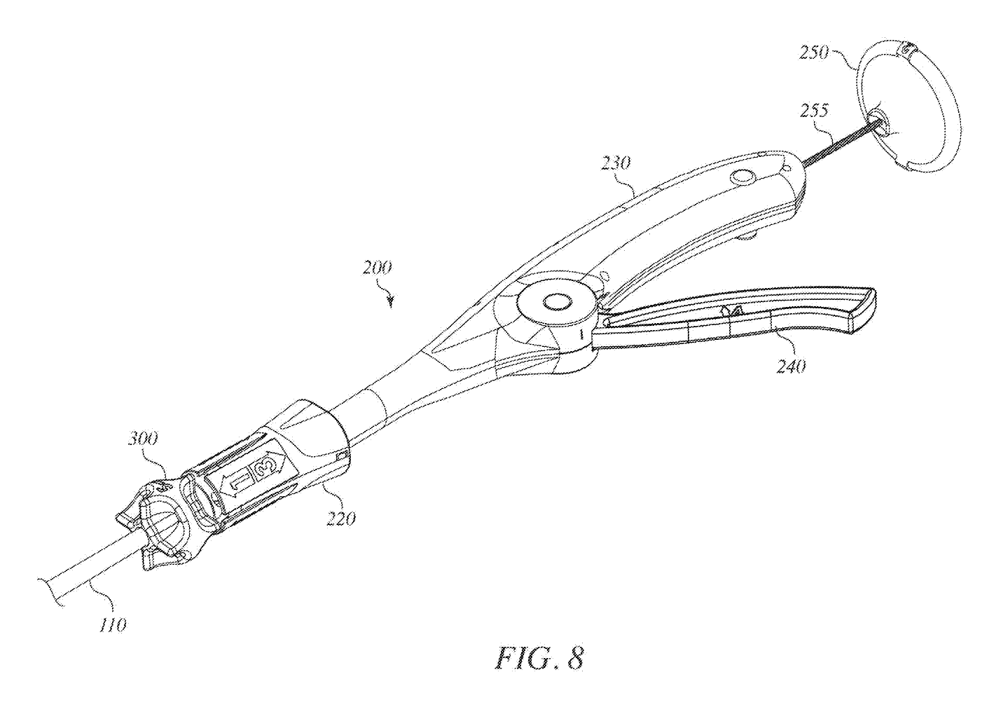

As shown in FIGS. 1 through 12E, a surgical instrument device according to the invention comprises a hollow housing, an activator or deployment means, and a flexible suture guide through which the suture is guided with, or extends with or is held within the flexible suture guide. The other end of the suture is contained within the hollow housing and having a knot therein or an anchor or a suture holder mechanism. The device includes a hollow housing through which the suture and flexible suture guide are housed in an unactivated state and then deployed into an activated state out of the distal end of the hollow housing, possibly out of a distal end cap on the distal end of the hollow housing, once activated by the activator or deployment means located on a proximal end of the hollow housing. The suture is placed around the target tubular vessel, organ or tissue and the severed, knotted and/or cut. The flexible suture guide is withdrawn back into the hollow housing then before or after it is withdrawn the suture is cut. Once the placed suture has been cut the inventive surgical device instrument is deactivated and retracted from the body cavity, unless further sutures are to be deployed within the body cavity at other locations such that the inventive surgical device instrument is deactivated, activated and repeated as needed.

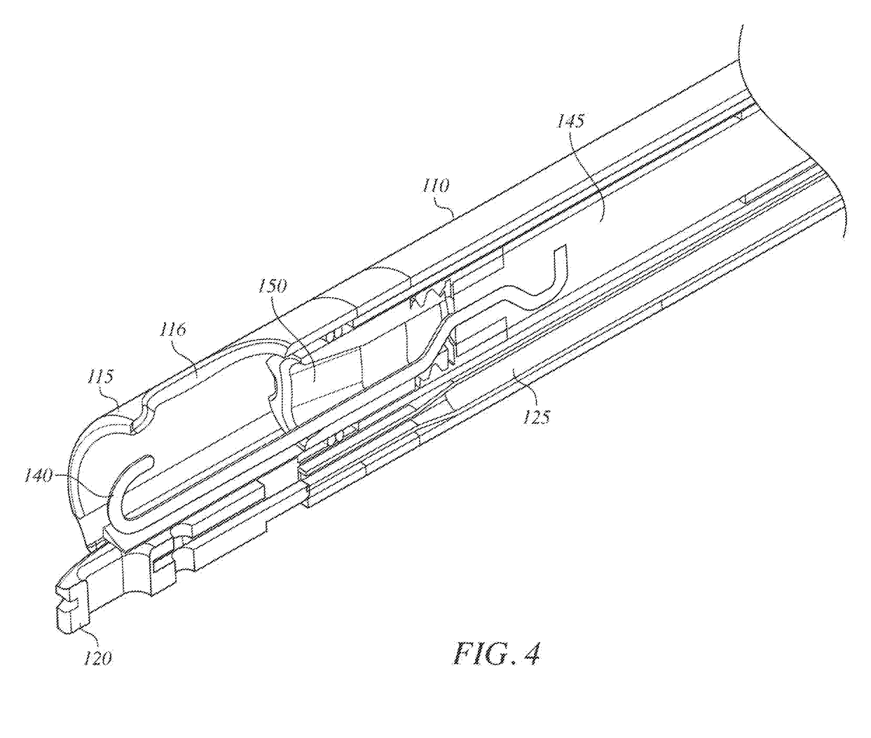

For the purposes of explanation and illustration, and not limitation, in accordance with the invention, exemplary embodiments of a surgical suturing device and method are illustrated in FIGS. 1 through 12A-E, with the inventive surgical suturing device shown show in the non-actuated state in FIGS. 1 through 3 and in a partially or full actuated state in FIGS. 4 through 11.

An embodiment of the present invention of the novel suturing device 100 is shown in FIGS. 1 through 11, wherein the suturing device 100 includes a hollow housing 110 connected on a distal end in one embodiment to a distal end cap 115 and connected on a proximal end to an actuating means in one embodiment such as a pistol style mechanism 200. The actuating means or deploying means may be triggered by a push rod, a plunger, a thumb grip, a pistol grip, a knob or other known actuating means and deployment mechanisms and is not limited to pistol style mechanisms.

As indicated above, the material and dimensions of the inventive suturing device 100 must be such that it can be inserted through a trocar or other opening on the body fascia during laparoscopic surgery. Typically, a hollow housing 110 or cannulas used in laparoscopic surgeries have inner diameters of from about 1.5 millimeter to about 50 millimeters, though smaller diameters of under 2.5 millimeters may be employed. The inventive surgical device may be self-inserting or it may be inserted within a trocar, which trocar diameter may affect the diameter of the housing 110. If self-inserting the distal end of the hollow housing 110 may be non-blunt shaped such as a cone, bull-nose of the like. In another embodiment a non-blunt shaped cap may be attached to the distal end of the hollow housing 110 or to the distal end cap 115.

The hollow housing 110 is hollow to accommodate various components of the inventive suturing device 100. The length of the hollow housing 110 may be affected by the location of the surgical site and location of the organ or tissue to be sutured by the inventive suturing device 100. The length of the hollow housing 110 may also be affected by the size of the patient such as an infant, child, adult, or obese patient. Other factors may affect the length, shape, curve or straightness, and diameter of the hollow housing 110.

In accordance with these examples, the inventive suturing device 100 includes a hollow housing 110 which may be a cannula or housing having a diameter, for example 3 mm or 10 mm, in a range of about 1 mm to about 100 mm, and with a length in a range of about 10 mm to about 1000 mm, preferably about 100 mm to about 600 mm. The hollow housing 110 may be straight or slightly curved. The hollow housing 110 may be curved or at an angle shape at one or more points along its horizontal axis. For example, the hollow housing 110 may be curved at a point with a curved portion located at a position on the hollow housing 110 approximately where the hollow housing 110 would be located outside of the body cavity, or at another location as determined by the size of the patient and distance within the body cavity and other factors. Other factors may be a curved or angle portion of the hollow housing 110 depending on the location of the target vessel or organ or tissue and its shape, such as curved around a tubular blood vessel at the distal end of the hollow housing 110 or having any known shape at the distal end or middle or proximal end so as to facilitate placement of the hollow housing 110 within the body cavity around organs or tissue. The hollow housing 110 (or cannula) of the inventive suturing device 100 may be comprised of any material compatible with the human body as a portion of it will be within the body cavity when in use. The hollow housing 110 is preferably comprised of a metal such as stainless steel and is preferably coated with a shrink wrap plastic such as shrinkable polyester, polyolefin, polyethylene, or polyvinyl chloride of a grade suitable for use in surgical procedures.

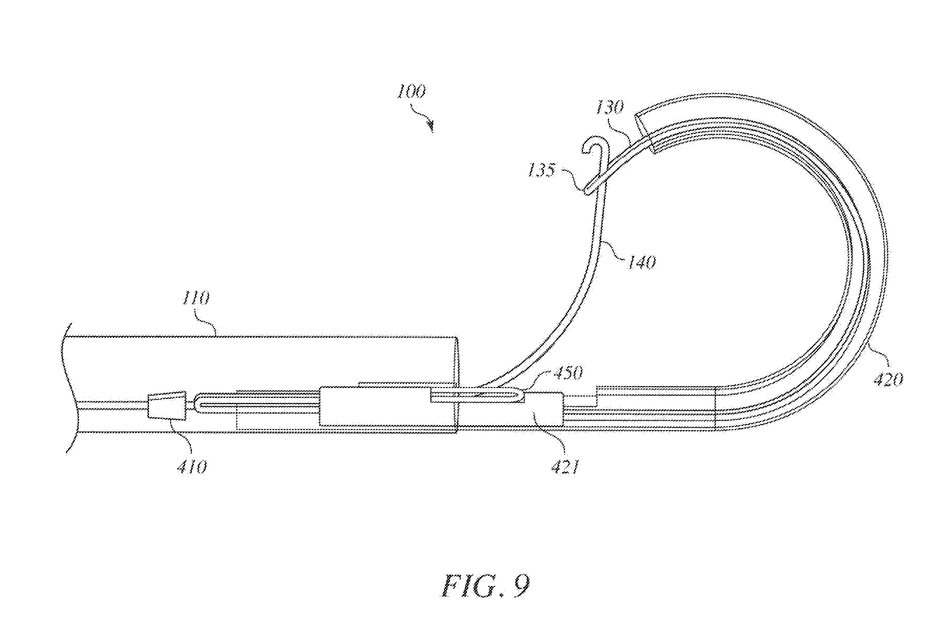

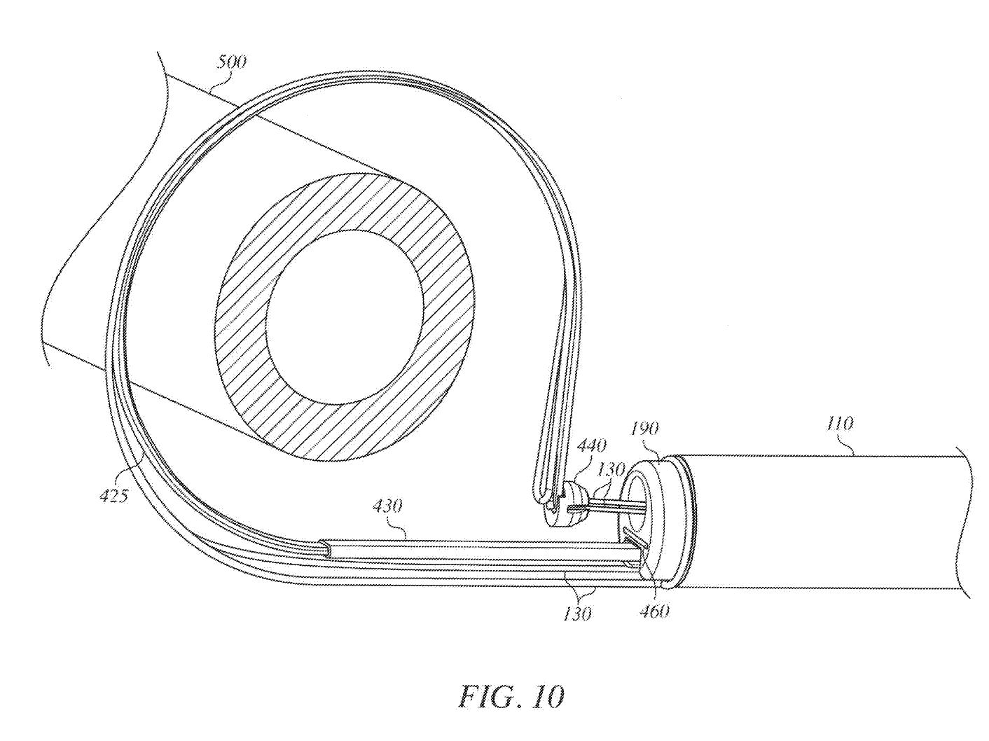

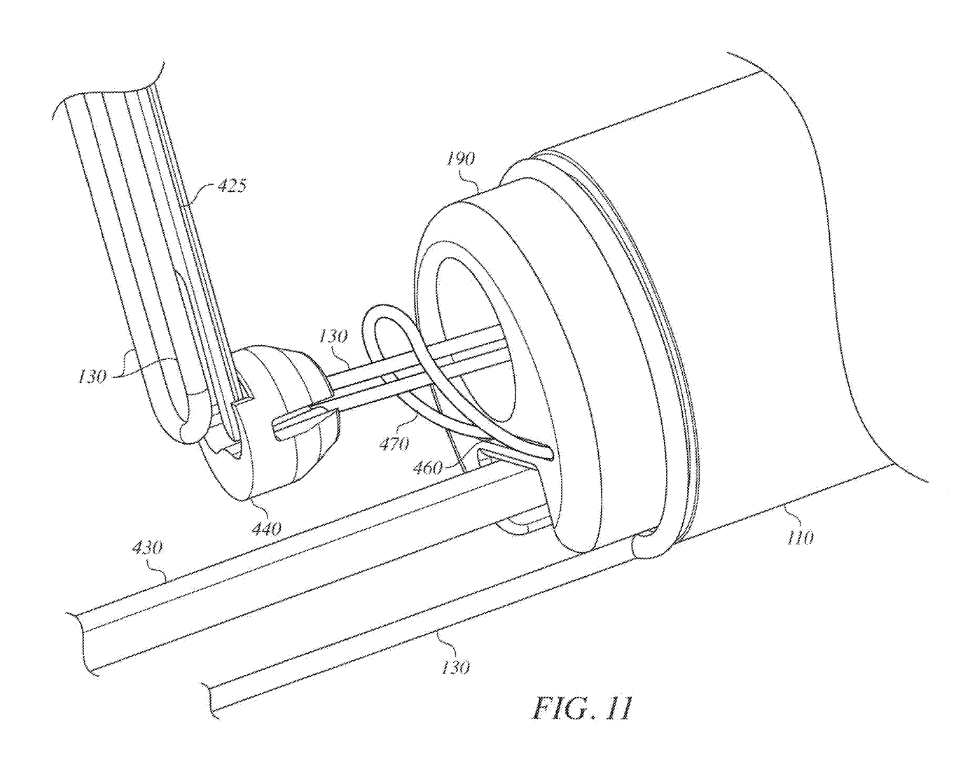

In one embodiment of the present invention the hollow housing 110 is connected on its distal end to a distal end cap 115 which includes an aperture 116. The distal end cap may be comprised of any material compatible with the human body as it will be within the body cavity when in use. The distal end cap 115 may the same or different material than the hollow housing 110. The distal end cap 115 is preferably comprised of a metal such as stainless steel and is preferably coated with a shrink wrap plastic such as shrinkable polyester, polyolefin, polyethylene, or polyvinyl chloride of a grade suitable for use in surgical procedures. The proximal portion of the distal end cap 115 is connected or joined to the distal end of the hollow housing 110 via any known joining or connecting means such as glue, welding, sodering, crimping or the like. The aperture 116 within the distal end cap 115 will house the distal end of the flexible suture guide 120 during use and the inventive method. The distal end cap 115 may be fully or almost fully open on the bias as shown in FIGS. 1-3 and 6, or partial open as shown in FIGS. 10 and 11. In one embodiment of the present invention as shown in FIG. 9, no distal end cap is employed.

The hollow housing 110 houses a flexible suture guide 120 which may include a preloaded suture 130. The flexible suture guide 120 may be curved sufficiently to curve around a tubular structure such as an appendix, cystic duct, cystic artery, blood vessel or the like. In certain instances the curve of the flexible suture guide 120 may travel an entire circle, a semi-circle, or may be between a semi-circle and complete circle. The material of the flexible suture guide must be made of any material compatible with the human body as a portion of it will be within the body cavity when in use. The flexible suture guide 120 may be comprised of an elastic wire or other elastic materials such as plastics, polymers and the like. In one embodiment the flexible suture guide 120 may be comprised of nitinol. The flexible suture guide 120 may be any known shape such as a “L”, angles such as a 90° or 45° angles or otherwise, or a combination of curves and angles relating to the location of the target vessel, tissue or organ. When a memory-shape material is used for the flexible suture guide 120 any single or combination of shapes may be used. An advantage of the present inventive suturing device 100 is that the flexible suture guide 120 shape is such that it is not dependent on the thickness of the vessel, tissue or organ to be sutured, compared to conventional staplers, clip appliers and other suturing devices.

In another embodiment of the present invention as shown in FIG. 9, the flexible suture guide is a hollowed curved housing 420. The hollowed curved housing 420 may be in the shape of a “J”, “U”, “C”, “S” or almost or fully enclosed “O” with a small opening to accommodate in use to place the hollowed curved housing 420 around the target vessel, tissue or organ for suturing. The hollowed housing 110 may be curved in any known angles such as without limitation between 150° to 300°. The hollowed curved housing 420 may be comprised of any material compatible with the human body as it will be operated within the body cavity. In another embodiment of the present invention the hollowed housing 110 may be curved initially but straightens out partially or fully as the suture 130 is pulled or tightened or knotted after placement on the outer diameter of the target Tubular vessel, tissue or organ.

In yet another embodiment of the present invention as shown in FIGS. 10 and 11, instead of a hollowed curve housing such as a cannula, the flexible suture guide 120 may be a curved elastic band 425 extending out of a guide tube 430, said guide tube 430 partially housed within the hollow housing 110 and extending out of the distal end cap 190.

In both embodiments as shown in FIGS. 9, 10 and 11 the hollowed housing 110 includes within a preloaded suture 130. The suture 130 may be comprised any material compatible with the human body as it will remain within the body cavity after the surgical procedure is completed. It is contemplated that the suture 130 would not be absorbable, but in certain embodiments it may be comprised of a material which absorbs into the body. The suture 130 may be comprised of silk, vicryl, davdelk, polypropylene, polymers or any other conventional thread or tying material. The term “suture” may also include plastic piece, wire, a ring, cable ties, or any other component that is capable of wrapping around a target vessel or other tubular structure and is not limited to a surgical thread or silk or the like.

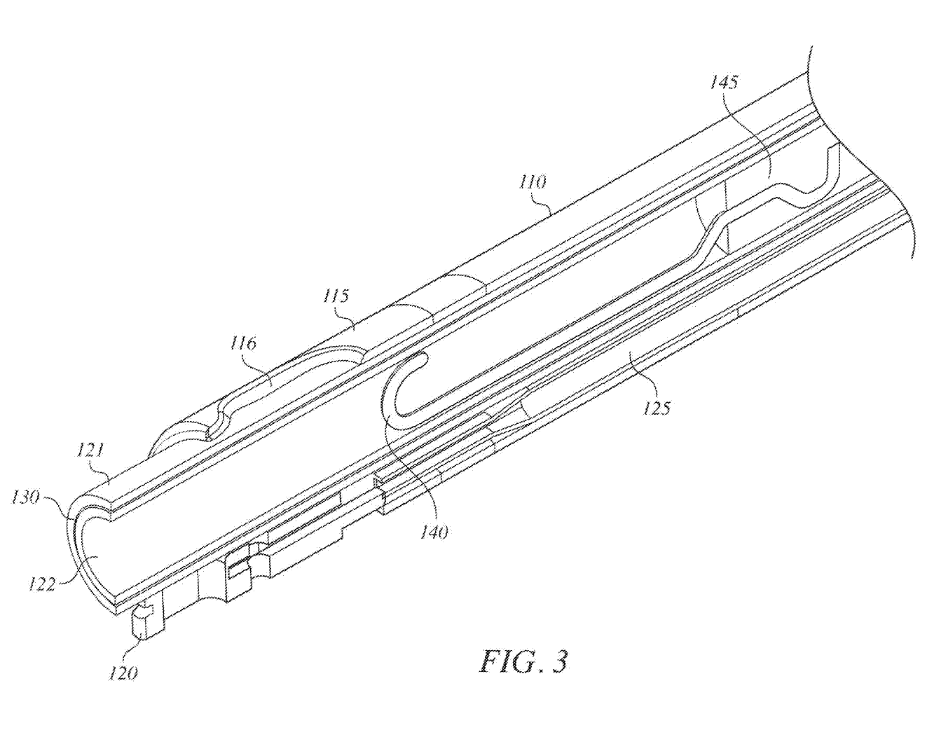

As shown in FIGS. 3, 4 and 5, the suture 130 is placed around, along or within (in different embodiments) the flexible suture guide 120 and one end of the suture coils around one of two tubes (121, 122) within the hollow housing 110. The first of the two tubes, the coil tube or outer tube 121, located within the hollow housing 110 pushes the coils of the suture 130 along the inner or second tube 122 after the suture 130 has been extended out of the hollow housing 110 along with the activated flexible suture guide 120 extended out of the hollow housing 110. The two tubes, the outer coil tube 121 and the inner coil tube 122 are deployed and extended out of the hollow housing 110 during use at such time as the suture 130 is being knotted and/or cut. In another embodiment the suture 130 is housed within the hollow housing 110 and is comprised of one long loop.

As shown in FIGS. 9, 10 and 11, the suture 130 within the hollow housing 110 may be preloaded in a looped formation. The suture 130 is folded in the lower portion to form two loops and is comprised of either one contiguous piece of thread, for instance, or may be joined together at the ends of the suture thread 130 by any known joining means, such as without limitation a crimp or other anchoring means. The joining means may be comprised of any material compatible with the human body such as without limitation a metal, plastic, polymer, biocompatible glue, or the like.

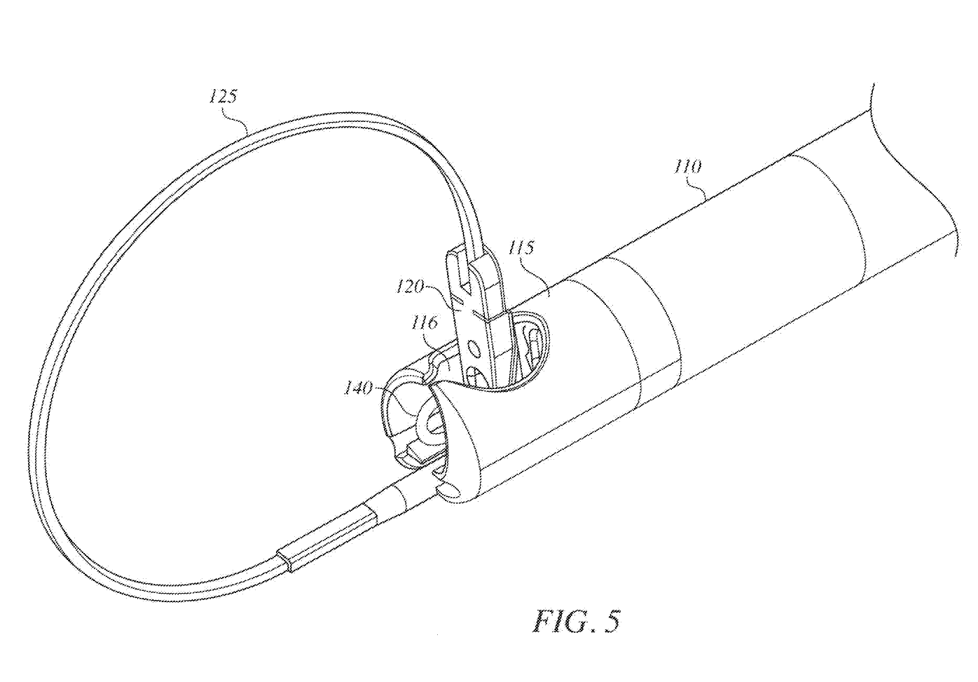

The suture 130 may be joined to a suture holder. In one embodiment of the invention the suture holder 150 (as shown in FIGS. 4 and 5) is rigid and may be comprised of plastic, polymers, metals and the like. In one embodiment of the invention the suture holder or anchor 150 is comprised of titanium. In this embodiment of the present invention the suture holder 150 is housed within the hollow housing 110 and attached to an activation means such as a plunger 145. The suture holder 150 may be attached to the activation means, plunger 145, by any known attachment means such as glue, welding, sodering, crimping or the like. In another embodiment of the present invention, the suture holder 150 may be a small slot to grab or tighten the suture. In yet another embodiment of the present invention the suture holder may be an anchor 440 as shown in FIGS. 10 and 11.

At the proximal end of the hollow housing 110 are the actuation means or deployment mechanisms. In one embodiment of the present invention as shown in FIGS. 1, 7 and 8, the actuation means is a pistol mechanism 200 in combination with a handle 220 and plunger 250. Other actuation means may be employed such as push rods, rods, slides, grips, handles, plungers, pull rods, thumb grips, and other known means.

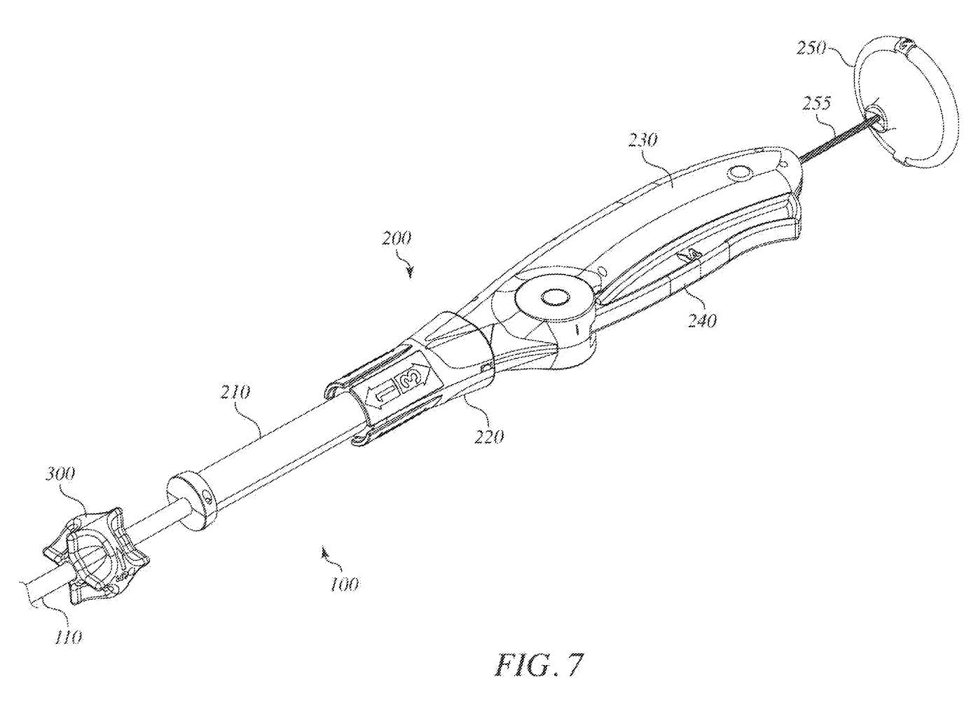

In one embodiment of the present invention the flexible suture guide 120 housed within the hollow housing 110 and is activated by an activation means. In one embodiment the activations means is a handle 220 which travels in a forward and retraction direction along the outer diameter of the hollow housing 110. In one embodiment of the present invention the handle 220 travels in a forward and backward direction along the handle hollow housing 210 as shown in FIGS. 1 and 7. In one embodiment of the present invention the handle 220 includes designation of numbers, such as “1” and “3” to indicate to the user or surgeon the order of steps of the inventive method. In use, the handle 220 is pushed in a forward direction towards the distal end of the hollow housing 110 (or 210) along the outer diameter of the hollow housing 110 and the flexible suture guide 120 is pushed in a forward direction out of the distal end the distal end of the hollow housing 110 (or distal end cap 115) of the hollow housing 110. In one embodiment, the handle 220 is connected within the hollow housing 110 to a proximal end of the flexible suture guide 120. The flexible suture guide 120 is pushed out of the distal end cap 115 and at some point begins to curve in a circular direction around the target vessel 500 such the that suture holder is forced into the aperture 116 on the distal end cap 115. The diameter of the flexible circle is contingent on the amount of flexible material used, such as for example and without limitation, nitinol. The flexible suture guide 120 could also form other shapes besides a circle such as a square, rectangle, oblong or any other shape.

The activation means in one embodiment of the present invention includes a hook 140 and a plunger mechanism 250. The hook 140 is housed within the hollow housing 110 and located at the distal end of the hollow housing 110. In use, once the rigid suture holder 150 is extended and housed in the aperture 116 of the distal end cap 115, the weight of the suture holder 150 presses the suture 130 against the flat portion of the hook 140. The hook 140 is then retracted back within the hollow housing 110 via an activation means. In one embodiment the activation means is a rod 145 with a plunger 250 or other type means to retract the hook 140. In one embodiment the plunger 250 has the number “2” on it to denote the second step of the inventive method. At the proximal end of the rod 145 small indentations to ratchet the retraction in smaller increments may be employed and to prevent the hook 140 from being moved in an opposite direction forward toward the distal end cap 115. At this point in use the hook 140 captures the suture 130 and tightens the suture 130 through the retraction of the hook 140.

In another embodiment of the present invention, as shown in FIG. 9, the hook 140 is moved out of the aperture 450 and hooks onto the upper portion of the suture 130 such as onto a crimp 135. At this point during the method and use the disposable suture tying end piece 420 is positioned around the target tubular object such as a vessel 500, and the suture 130 is in the unactivated position. The hook 140 movement is then reversed and the hook 140 is pulled back into the aperture 450 and into the flat portion 421 of the preloaded disposable suture tying end piece 420 (the flexible suture guide 120) with such movement creating a knot of the suture 130. As the knot tightens the hook 140 retracts further into the hollow housing 110 and the preloaded disposable suture tying end piece 420 may either be released out of the hollow housing 110 or if the preloaded disposable suture tying end piece 420 is of a flexible material it may straighten out once the tension is released along with the lower looped portion of the suture 130 being released. Other known release mechanisms may be employed.

The activation means in one embodiment of the present invention also includes a pistol style grip including a housing 230 and a trigger 240. The pistol housing 230 houses the rod 145 and a ratchet mechanism (not shown) which is connected to the trigger 240. The trigger 240 operates in use to push the anchor 150 and coils 160 of the suture 130 against the target vessel 500 until the suture 130 is tightened to the surgeon's satisfaction. In one embodiment the trigger 240 has the number “4” on it to denote the fourth step of the inventive method.

Within the hollow housing 110 may be disposed one or more hollow coiling tubes 121, 122. In one embodiment of the present invention there are two coiling tubes 121, 122 within the hollow housing 120. The first outer tube or coil tube 121 moves the coils of the suture 130 in a forward and backward direction at the proximal end of the suture 130. The coils of the suture 130 are located on the outer diameter of the second or inner tube 122. The coils of the suture 130 remain in an unactivated state within the hollow housing 110 and are moved in a downward direction prior to activating the knot or anchor 150 of the suture 130 to tie or knot the suture 130 around the target vessel 500. The outer coil tube 121 is activated by the trigger 240 through a ratchet mechanism or other gear or other activation means. In another embodiment of the present invention a locking mechanism (not shown) may lock the outer coil tube 121 so that it cannot be retracted back into the hollow housing 110. The ratchet mechanism connected to the trigger 240 may also include an audible clicking so the surgeon can gauge the number of clicks and to slow down the movement of the outer coil tube 121.

The second inner coil tube 122 houses and moves the anchor mechanism 150 from an unactivated state within the hollow housing 110 into an activated state to anchor the suture 130 when in use. Many possible configurations may be used for the anchor mechanism and two are shown in FIGS. 3 and 4. In FIG. 3 the anchor mechanism 150 is a nut and screw configuration.

The coils of the suture 130 rest on top of the inner coil tube 122 and the coil tubes 121, 122 are same size so that distal end of the outer coil tube 121 pushes the coils 131 out along top or outer diameter of the inner coil tube 122. The outer or coil tube 121 and second inner tube 122 travel together and typically neither will extend out of the hollow housing 120 farther than the other tube. The coil tubes 121, 122 travel to a distance outside of the hollow housing 120 such that the anchor 150 faces the target vessel.

In one embodiment the anchor 150 has a nut and screw configuration as shown in FIG. 4. The screw has an opening corresponding with an opening in the nut. The nut and screw are activated through the rotational handle 300 which is connected at proximal end of the outer coil tube 121. The rotational handle 300 has an aperture (not shown) through which the hollow housing 110 is inserted during production of the inventive surgical suturing device 100 such the rotational handle 300 rides along the outer diameter of the hollow housing 110. As the rotational handle 300 is rotated in the chosen direction the distal end of the outer coil tube 121, which is coupled to the nut, the outer coil tube 121 rotates and likewise rotates the nut so that it is locked into the screw thus locking the anchor 150. In one embodiment the rotational handle 300 has the number “5” on it to denote the fifth step of the inventive method and also has an arrow pointing in the rotational direction to activate and rotate the nut so that it is locked into the screw to secure and lock the anchor 150.

Turning to the inventive method of the present invention, the suturing device 100 is inserted into a body cavity and located near the target tubular vessel, organ or tissue. In one embodiment of the present inventive method of use as shown in FIGS. 1 through 8, the handle 220 is pushed in a direction along the outer diameter of the hollow housing 110 and the flexible suture guide 120 is pushed in a forward direction out of the distal end cap 115 of the hollow housing 110. At some point the flexible suture guide 120 begins to curve in a circular direction around the target vessel 500 such the that distal end of the flexible suture guide 120 is forced into the aperture 116 on the distal end cap 115. Other activation means besides a handle 220 may be employed so as to activate the flexible suture guide 120 such as without limitation grips, plungers, push or pull rods and the like. The suture is moved from an inactivated state either within or alongside the flexible suture guide 120 and placed around the outer diameter of the target tubular vessel, tissue or organ.

The hook 140 is activated and grabs the suture 130 through various means and methods and pulls or tightens the suture 130 around the outer diameter of the target tubular vessel, tissue or organ. The hook 140 is then retracted back within the hollow housing 110 via an activation means. In one embodiment the activation means is a rod 145 with a plunger 250 or other type means to retract the hook 140. The rod 145 is slowly retracted and the surgeon has an audible clicking to measure the retraction through the indentations on the proximal end of the rod 145. Other activation means besides a rod 145 and/.or plunger 250 may be employed so as to activate the hook 140 such as without limitation grips, handles, plungers, push or pull rods and the like.

The surgeon next reverses direction of the handle 220 and the flexible suture guide 120 is reacted back into the hollow housing 110. At this point in the inventive method of use the suture 130 is in place around and partially tightened around the target vessel 500.

The next step is for the surgeon to activate the anchor mechanism. The surgeon ratchets the trigger 240 which pushes the coils 131 of the suture 130 and pushes the coiling tubes 121, 122 and the anchor 150 out of the hollow housing 110 (past the distal end cap 115 if included in the embodiment of the suturing device 100) and near or against the target vessel 500 until the suture 130 is either knotted with the knot or the suture 130 is closed via the anchor 150, such that the suture 130 is tightened to the surgeon's satisfaction. The surgeon has an audible signal as to the ratcheting of the trigger 240. Other known activation means besides a trigger 240 may be employed so as to activate the coils and anchor 150 such as without limitation grips, handles, plungers, push or pull rods and the like.

In an embodiment of the present invention employing a nut and screw combination anchor 150, the rotational knob 300 is rotated so as to lock the nut and the screw to form the locked or secured anchor 150 on the suture 130.

The portion of the suture 130 located at the knot or the anchor 150 is then cut or severed but the surgeon. In one embodiment of the present invention a cutting means, such as a blade is included on the distal end cap 115. A further option is choosing the tensile strength of the suture 130 related to the retraction rate of the hook 140 so as to cause the anchored suture end to break rather than being cut. Other cutting or severing means may be employed and added to the inventive device 100.

Other possible cutting or severing means may be used. For instance, a piece of plastic (not shown) may be included within the distal end cap and configured with a smaller neck position connected to the suture 130 so that a calibrated force when the hook 140 is retracted within the hollow housing 110 would break the plastic portion and thus sever the suture portion located within such plastic piece.

Yet another cutting means 470 is shown in FIG. 11. An aperture or opening 460 is included in the bottom portion of the distal end cap 115 of the hollow housing 110 through which a suture cutter 470 may be activated so as to cut the suture 130 after the suture 130 has been knotted around the target object or vessel 500. The suture cutter 470 may be a wire, comprised of any conductive material such as tungsten or other high resistance and high melting temperature materials, which is suture cutter 470 is connected to an energy source such as a battery or electricity. Upon activation, the suture cutter 470 is deployed out of the opening 460, activated with an energy source, and cuts the suture 130 via such activation energy, such as heat. Thus the surgeon needs only one device and not a separate scissors, cauterizer, knife or the like.

In another embodiment of the inventive method of use employing the suturing device 100 as shown in FIGS. 10 and 11, the inventive method of use begins with the looped suture 130. The suture 130 is folded over on itself so that the lower portion forms two loops and in this embodiment the suture 130 thread ends are joined via a crimp 135 at an upper portion location of the suture 130. The suture 130 is preloaded into the hollowed curved housing 420 with the two smaller loops within the flat portion 421 of the hollowed curved housing 420 which is located within the distal end of the hollow housing 110. An upper portion of the preformed suture 130 (optionally including the joining means such as the crimp 135) extends out of the distal end of the hollowed curved housing 420. This upper portion of the suture 130 forms a ring or eye which can be grabbed and pulled to release the lower double looped portion of the suture 130 in use. A lock hub 410 may also be included which releases upon actuation to release the lower portion of the suture 130. In use the suturing device 100 is inserted into a body cavity and the distal end of the hollow curved housing 420 is placed at a location around the outer diameter of the target tubular organ 500 (for illustrative purposes only and not limited to just a tubular organ). Once the knot (not shown) has been sufficiently tightened by the surgeon, an optional retaining means, such as a fastener, cone, barb, anchor or the like may be placed on the knot for further support.

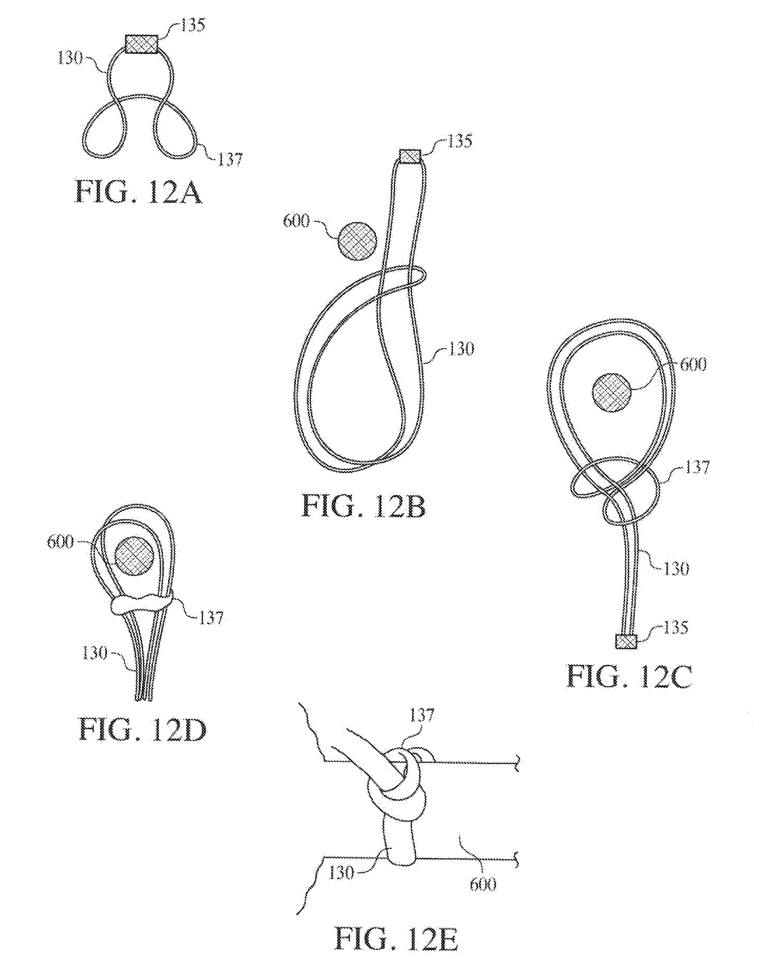

In one embodiment of the present invention, a suture anchor 440 is included loaded onto the suture 130 as shown in FIGS. 11 and 12. The suture 130 is preloaded with the guide 425 with the two smaller loops within the flat portion 421 of the flexible curved guide 425 which is located within the distal end of the hollow housing 110. An upper portion of the preformed suture 130 (optionally including the joining means such as the crimp 135) extends out of the distal end of the hollowed curved guide 425. This upper portion of the suture 130 forms a ring or eye which can be grabbed and pulled to release the lower double looped portion of the suture 130 in use. A lock hub 410 may also be included which releases upon actuation to release the lower portion of the suture 130. In use the suturing device 100 is inserted into a body cavity and the distal end of the hollow curved housing 420 is placed at a location around the outer diameter of the target tubular organ 500 (for illustrative purposes only and not limited to just a tubular organ). Once the knot (not shown) has been sufficiently tightened by the surgeon, an optional retaining means, such as a fastener, cone, barb, anchor or the like may be placed on the knot for further support.

In yet another embodiment of the present invention as shown in FIGS. 10 and 11, instead of a hollowed curve housing such as a cannula, the flexible suture guide 120 may be a curved elastic band 425 extending out of a guide tube 430, said guide tube 430 partially housed within the hollow housing 110 and extending out of the distal end cap 190.

The surgeon may then cut the suture 130 located above the knot and optional retaining means 470 or 440). A suture is thus obtained on a tubular structure using only one device. The suturing device 100 is then removed from the body cavity.

In the embodiment shown in FIGS. 9, 10 and 11, it is contemplated that the preloaded disposable suture tying end piece 420 is disposable while the hollow housing 110 and proximal end of the device 100 is either reusable or also disposable. In other embodiments of the invention the preloaded suture tying end piece may be reusable and capable of reloading with the suture 130.

Another embodiment of the present inventive method and device is shown in FIGS. 12A through 12E using an embodiment of the device as shown in FIG. 9 and wherein a preloaded knot 137 (unactivated and not tightened) is shown in FIG. 12A. The knot 137 has the bottom section of the suture lopped over itself as shown in FIG. 12A, though other variations and embodiments of the knot structure may be used. The knot 137 may be located at one end of the suture 130. The inventive suturing device 100 may be used in various surgical procedures such as but not limited to suturing an appendix 600. In use the suturing device 100 is inserted into a body cavity and the distal end of the flexible suture guide 420 is placed at a location around the outer diameter of the appendix 600 (for illustrative purposes only and not limited to just the appendix organ). The suture 130 may include a crimp 135 at one end of the suture loop and preloaded knot 137. One embodiment of a suture 130 is shown in FIG. 12B with the knot formed as the folded suture loops on itself. When the flexible suture guide 420 is placed at a location around the outer diameter of the appendix 600 the suture 130 may be partially extended out of a distal end of the flexible suture guide 420. The hook 140 is moved out of the aperture 150 and hooks onto the upper portion of the suture 130 such as onto the crimp 135. The hook 140 movement is then reversed and the hook 140 is pulled back into the aperture 450 and into the flat portion 421 of the preloaded flexible suture guide 420 with such movement creating a knot 137 of the suture 130. The knot 137 formation is shown in FIGS. 12C and 12D. As the knot 137 tightens the hook 140 retracts further into the hollow housing 110 and the flexible suture guide 420 may either be released out of the hollow housing 110 or if the flexible suture guide 420 is of a flexible material it may straighten out once the tension is released along with the lower looped portion of the suture 130 being released. Other known release mechanisms may be employed.

Once the knot 137 has been sufficiently tightened by the surgeon, an optional retaining means 410, such as a fastener, cone, barb, anchor or the like may be placed on the knot for further support. In one embodiment of the present invention, a suture anchor is included loaded onto the suture 130.

The surgeon may then cut the suture 130 located above the knot 137 (and optional retaining means 170 or 240). A suture is thus obtained on a tubular structure as shown in FIG. 12E and it has been accomplished using only one device. The suturing device 100 is then removed from the body cavity. It is contemplated that the flexible suture guide 420 is disposable while the hollow housing 110 and proximal end of the device is either reusable or also disposable. In other embodiments of the invention the preloaded flexible suture guide may be reusable and capable of reloading with the suture 130.

Advantages of the inventive suturing device 100 include the configuration such that it is capable of suturing a tubular structure, which reduces the need for a second device being used such as a grasper or other surgical instrument. Accordingly the surgical process for suturing can be simplified by using the inventive suturing device 100 and the time and cost for the surgery can be reduced.

Thus, the suturing device 100 may reduce complications, surgical processes, time and cost. Further the present invention provides a new suturing device which permits the suturing operation to be carried out by the surgeon without the necessity of resorting to a second device or even an assistant. Additionally, the inventive suturing device 100 is not dependent on the thickness of the area to be sutured or the diameter of a target object to be sutured, whether it is tubular or otherwise. Further, the inventive suturing device 100 may suture and cuts the suture in the same device.

Many possible combinations could be within the suturing device, the methods of use, and the kit or system of the present invention. While a tubular vessel, tissue or organ has been discussed the suturing device may be used in non-tubular locations within the body cavity as well. Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

It will be understood that the invention is not restricted to the above described exemplifying embodiments thereof and that several modifications are conceivable within the scope of the following claims.

We claim:

1. A surgical assembly for suturing a tubular vessel or organ, comprising:
a hollow housing including a suture housed within the hollow housing;
a flexible suture guide configured to be placed around an outer diameter of a tubular vessel or organ and connected to a distal end of the hollow housing and connected on a proximal end to an activator; and
an activator that is configured to impart a force onto the suture such that the suture tightens and forms a knot at a pre-knotted proximal end of the suture.

2. The surgical assembly of claim 1 further comprising a cutting means to cut the suture at a location near the tightened knot.

3. The surgical assembly of claim 2 wherein the cutting means may be a knife, blade, cap, wire, or electric material.

4. The surgical assembly of claim 3 wherein the cutting means is a blade.

5. The surgical assembly of claim 1 wherein the flexible suture guide is hollow and contains a portion of the suture.

6. The surgical assembly of claim 1 wherein the activator may be a hook, push-pull mechanism, or a rod mechanism.

7. A surgical assembly for suturing a tubular vessel or organ, comprising:
a hollow housing having an aperture on a horizontal plane;
a suture housed within the hollow housing;
a flexible suture guide within the hollow housing and connected on a proximal end to an activator,
a hook within the hollow housing connected on a proximal end to a rod; and
an activator that is configured to impart a force onto the proximal end of the flexible suture guide to urge the portion of the flexible distal end of the hollow housing and flexibly bend in a direction an so that a distal end of the flexible suture guide falls within the aperture on the hollow housing and to be released from the aperture and retracted back into the hollow housing,
wherein the suture is looped over the sides of the flexible suture guide and coiled within the hollow housing.

8. The surgical assembly of claim 7 further comprising a severing means to sever the suture at a location near the tightened knot.

9. The surgical assembly of claim 8 wherein the severing means may be a knife, blade, cap, wire, or electric material.

10. The surgical assembly of claim 9 wherein the severing means is a blade.

11. The surgical assembly of claim 7 wherein the flexible suture guide is hollow and contains a portion of the suture.

12. The surgical assembly of claim 7 wherein the activator may be a hook, push-pull mechanism, or a rod mechanism

13. The surgical assembly of claim 7 further comprising an anchor attached at a proximal end of the suture.

14. The surgical assembly of claim 13 wherein the anchor is comprised of a nut and screw configured to rotate and lock so as to anchor the proximal end of the suture.

15. The surgical assembly of claim 7 further comprising a sharp means at a distal end of the hollow housing with a shape in a form selected from the group consisting of a cone, bull-nose, hook, curve, triangle and combinations therein.

16. A method of suturing a tubular vessel or organ, comprising the steps of:
inserting a suturing device having a hollow housing, a flexible suture guide, a suture have a pre-formed knot on its proximal end and an activator and a severing means, within the fascia and into a body cavity located in the vicinity of a tubular vessel or organ;
activating the activator such that the suture is placed around the outer diameter of the tubular vessel or organ;
tightening the suture around the outer diameter of the tubular vessel or organ to form a knot; and
severing the suture at a location near the knot using the severing means of the suturing device.

17. The method according to claim 16 wherein the severing means may be a knife, blade, cap, wire, or electric material.

18. The method according to claim 16 wherein the flexible suture guide is hollow and contains a portion of the suture.

19. The method according to claim 16 wherein the activator may be a hook, push-pull mechanism, or a rod mechanism.

20. The method according to claim 16 wherein the suture includes an anchor attached at a proximal end of the suture, the anchor including a nut and screw configured to rotate and lock so as to anchor the proximal end of the suture and the method further comprising the step of activating the anchor at the proximal end of the suture prior to severing the suture.
